(19) United States
(12) Patent Application Publication
(10) Pub. No.: US 2018/0164248 A1
(43) Pub. Date: Jun. 14, 2018

(54) GAS DETECTION DEVICE

(71) Applicant: Toyota Jidosha Kabushiki Kaisha, Toyota-shi Aichi-ken (JP)

(72) Inventors: Kazuhisa Matsuda, Susono-shi Shizuoka-ken (JP); Kazuhiro Wakao, Susono-shi Shizuoka-ken (JP); Keiichiro Aoki, Sunto-gun Shizuoka-ken (JP)

(21) Appl. No.: 15/835,678

(22) Filed: Dec. 8, 2017

(30) Foreign Application Priority Data
Dec. 13, 2016 (JP) 2016-241488

(51) Int. Cl. G01N 27/48; G01N 33/00

(52) U.S. Cl. CPC G01N 27/48; G01M 15/102; G01N 33/0036; G01N 33/0042

(57) ABSTRACT

A gas detection device includes: an electrochemical cell, a voltage applying unit, a current detecting unit that detects an output current which is a current flowing between a first electrode and a second electrode of the electrochemical cell; a voltage control unit that performs a step-up process of gradually stepping up an application voltage from a first voltage to a second voltage and performs a step-down process of gradually stepping down the application voltage to a third voltage after the step-up process has been completed; and a measurement unit. The voltage control unit sets the second voltage to a voltage lower than a voltage at which a rapid increase of the output current occurs due to reductive decomposition of water contained in the exhaust gas when the application voltage increases gradually.

### CROSS-REFERENCE TO RELATED APPLICATIONS

This application claims priority to Japanese Patent Application No. 2016-241488 filed on Dec. 13, 2016, the entire contents of which are incorporated herein by reference in its entirety.

### BACKGROUND

### 1. Technical Field

The disclosure relates to a gas detection device that determines whether a sulfur oxide concentration in an exhaust gas (a sample gas) discharged from an internal combustion engine is higher than a predetermined value or detects a sulfur oxide concentration in the exhaust gas.

### 2. Description of Related Art

An air-fuel ratio sensor (hereinafter also referred to as an “A/F sensor”) that acquires an air-fuel ratio (A/F) of an air-fuel mixture in a combustion chamber based on a concentration of oxygen (O₂) contained in an exhaust gas has been widely used to control an internal combustion engine. A limit current type gas sensor is known as such a type of air-fuel ratio sensor.

A SOx concentration detection device (hereinafter also referred to as a “conventional device”) that detects a concentration of sulfur oxide (hereinafter also referred to as “SOx”) in a sample gas by employing the limit current type gas sensor has been proposed (for example, see Japanese Unexamined Patent Application Publication No. 2015-17931 (JP 2015-17931 A)).

The conventional device includes a pump cell (an electrochemical cell) using an oxygen pumping effect of a solid electrolyte with oxygen ion conductivity. The conventional device generates oxide ions (O²⁻) by applying a voltage to the pump cell to decompose gas components (oxygen or oxygen-containing components (such as SOx and H₂O)) in a sample gas. The conventional device detects characteristics of a current (hereinafter also referred to as an “output current”) flowing between electrodes of the pump cell due to migration of oxide ions, which have been generated by decomposition of the gas components, between the electrodes (an oxygen pumping effect).

Specifically, when a SOx concentration is detected, the conventional device performs a step-up process of gradually stepping up an application voltage of the pump cell from 0.4 V to 0.8 V and then performs a step-down process of gradually stepping down the application voltage from 0.8 V to 0.4 V. The conventional device calculates the SOx concentration using a difference between a “reference current” which is an output current at a time point at which the application voltage reaches 0.8 V and a “peak value” which is a minimum value of the output current in a period in which the application voltage decreases from 0.8 V to 0.4 V.

### SUMMARY

Appearance of the peak value (that is, a temporary decrease of the output current) results from migration of oxide ions, which have been generated when sulfur (S) adsorbed (deposited) on one (a negative electrode) of the electrodes of the pump cell is changed to SOx by reoxidation, between the electrodes. Adsorption of sulfur on the negative electrode is caused with reductive decomposition of SOx which is caused when the application voltage is higher than a decomposition start voltage of SOx.

In order to accurately detect a SOx concentration, it is preferable that the peak value become smaller as the SOx concentration in an exhaust gas becomes larger. Accordingly, sulfur needs to be adsorbed on the negative electrode by increasing the application voltage to as high a voltage as possible in a range higher than the decomposition start voltage of SOx and reductively decomposing a sufficient amount of SOx corresponding to the SOx concentration.

However, when the application voltage is higher than a decomposition start voltage of water, water is reductively decomposed in the exhaust gas in addition to SOx. A water concentration in an exhaust gas is much higher than the SOx concentration. Accordingly, when the application voltage becomes higher than the decomposition start voltage of water, the output current increases rapidly due to reductive decomposition of a large amount of water. Thereafter, when the application voltage becomes lower than the decomposition start voltage of water, the output current decreases significantly. This phenomenon in which the output current decreases significantly is estimated to be caused due to a rapid decrease in an amount of oxide ions migrating between the electrodes of the pump cell when the application voltage is changed from a voltage higher than the decomposition start voltage of water to a voltage lower than the decomposition start voltage of water. A “change of the output current caused when sulfur adsorbed on the negative electrode is re-oxidized” is affected by the significant decrease of the output current and thus there is a likelihood that the peak value will change and detection accuracy of the SOx concentration will deteriorate.

The disclosure provides a gas detection device that can accurately determine whether a sulfur oxide concentration in an exhaust gas is higher than a predetermined threshold value or detect the sulfur oxide concentration in the exhaust gas by excluding an influence of water in the exhaust gas as much as possible.

A gas detection device according to an aspect of the disclosure (hereinafter also referred to as the “invented device”) includes an electrochemical cell, a voltage applying unit, a current detecting unit, a voltage control unit, and a measurement unit.

The electrochemical cell includes a solid electrolyte which has oxide ion conductivity, of which a first surface is exposed to an exhaust gas of an internal combustion engine introduced via a diffusion resistor, and of which a second surface other than the first surface is exposed to atmospheric air, a first electrode which is disposed on the first surface of the solid electrolyte, and a second electrode which is disposed on the second surface of the solid electrolyte.

The voltage applying unit applies an application voltage, which has a positive value when a potential of the second electrode is higher than a potential of the first electrode, between the second electrode and the first electrode.

The current detecting unit detects an output current which is a current flowing between the first electrode and the second electrode.

The voltage control unit performs a step-up process of gradually stepping up the application voltage from a predetermined first voltage lower than a specific voltage which is a voltage at which reductive decomposition of sulfur oxide contained in the exhaust gas occurs to a predetermined second voltage higher than the specific voltage and performs a step-down process of gradually stepping down the application voltage to a predetermined third voltage lower than the specific voltage after the step-up process has been completed.

The measurement unit determines whether a sulfur oxide concentration in the exhaust gas is higher than a predetermined value or detects the sulfur oxide concentration in the exhaust gas based on a specific parameter which has a correlation with a degree of change of the output current caused due to return of sulfur generated by reductive decomposition of the sulfur oxide to sulfur oxide on the first electrode during the step-down process.

The voltage control unit sets the second voltage to a voltage lower than a voltage at which a rapid increase of the output current occurs due to reductive decomposition of water contained in the exhaust gas when the application voltage increases gradually.

For example, the specific parameter is a difference between an “output current (a reference current) corresponding to a predetermined application voltage” which is acquired before the step-up process is started and a “minimum value of the output current (a current minimum value) when the application voltage is lower than a decomposition start voltage of SOx” which is acquired during the step-down process. That is, the specific parameter may be a difference between a current value which is acquired before reductive decomposition of SOx occurs and a current value which is acquired when reoxidation of SOx occurs. In this case, it is determined that the SOx concentration becomes higher as the specific parameter becomes larger. Alternatively, it is determined that the SOx concentration is higher than a predetermined value when the specific parameter is larger than a predetermined threshold value.

A voltage at which a rapid increase of the output current occurs due to electrolysis of water contained in the exhaust gas when the application voltage increases gradually is also referred to as a “decomposition activation voltage of water.” The second voltage is set to be lower than the decomposition activation voltage of water. Accordingly, according to the invented device, it is possible to prevent a “change of the output current (a reoxidation current change) caused by returning of sulfur produced by reductive decomposition of sulfur oxide to sulfur oxide during the step-down process” from being affected by a rapid increase of the output current in the step-up process due to reductive decomposition of water and a significant decrease of the output current in the step-down process to change the specific parameter and detection accuracy of the SOx concentration from deteriorating. Accordingly, according to the invented device, it is possible to accurately determine whether the sulfur oxide concentration in the exhaust gas is higher than a predetermined threshold value or detect the sulfur oxide concentration in the exhaust gas.

In the aspect, the gas detection device may further include an air-fuel ratio acquiring unit that acquires an air-fuel ratio of an air-fuel mixture in a combustion chamber of the internal combustion engine, and the voltage control unit may set the second voltage to become higher as the air-fuel ratio of the air-fuel mixture in the combustion chamber of the internal combustion engine acquired by the air-fuel ratio acquiring unit becomes higher.

The decomposition activation voltage of water becomes higher as the air-fuel ratio of the air-fuel mixture in the combustion chamber of the internal combustion engine becomes higher (see Points P3a to P3c in FIG. 7). Accordingly, when the second voltage is set to become higher as the air-fuel ratio becomes higher in a range which is lower than the decomposition activation voltage of water, it is possible to reductively decompose a sufficient amount of SOx corresponding to the SOx concentration when the application voltage is higher than the decomposition start voltage of SOx and to prevent the reoxidation current change from being affected by a rapid increase of the output current in the step-up process due to reductive decomposition of water and a significant decrease of the output current in the step-down process. Accordingly, according to the aspect, it is possible to accurately determine whether the sulfur oxide concentration in the exhaust gas is higher than a predetermined threshold value or detect the sulfur oxide concentration in the exhaust gas.

In the aspect, the gas detection device may further include a temperature acquiring unit that acquires a temperature of the electrochemical cell, and the voltage control unit may set the second voltage to become lower as the temperature of the electrochemical cell acquired by the temperature acquiring unit becomes higher.

The decomposition activation voltage of water becomes lower as the temperature of the electrochemical cell becomes higher (see Points P4a to P4c in FIG. 8). Accordingly, when the second voltage is set to become lower as the temperature of the electrochemical cell becomes higher in a range which is lower than the decomposition activation voltage of water, it is possible to reductively decompose a sufficient amount of SOx corresponding to the SOx concentration when the application voltage is higher than the decomposition start voltage of SOx and to prevent the reoxidation current change from being affected by a rapid increase of the output current in the step-up process due to reductive decomposition of water and a significant decrease of the output current in the step-down process. Accordingly, according to the aspect, it is possible to accurately determine whether the sulfur oxide concentration in the exhaust gas is higher than a predetermined threshold value or detect the sulfur oxide concentration in the exhaust gas.

In the above description, for the purpose of easy understanding of the disclosure, names and/or reference signs which are used in an embodiment which will be described later are added in parentheses to elements of the disclosure corresponding to the embodiment. However, the elements of the disclosure are not limited to the embodiment defined by the names and/or reference signs. Other objects, features, and advantages of the disclosure will be easily understood from the following description of the embodiment of the disclosure which will be described below with reference to the accompanying drawings.

### BRIEF DESCRIPTION OF THE DRAWINGS

Features, advantages, and technical and industrial significance of exemplary embodiments of the disclosure will be described below with reference to the accompanying drawings, in which like numerals denote like elements, and wherein:

FIG. 1 is a schematic diagram of an internal combustion engine (a present internal combustion engine) to which a gas detection device according to an embodiment of the disclosure (a present detection device) is applied;

FIG. 2 is a schematic cross-sectional view of an element portion of the present detection device;

FIG. 3 is a timing chart illustrating an outline of an operation of the present detection device;

FIG. 4A is a diagram illustrating a change of an output current with respect to a change of an application voltage when an upper limit voltage is lower than an activation voltage of water;

FIG. 4B is a diagram illustrating a change of the output current with respect to a change of the application voltage when the upper limit voltage is higher than the activation voltage of water;

FIG. 5A is a diagram illustrating an example of a change of the application voltage with the lapse of time;

FIG. 5B is a diagram illustrating another example of the change of the application voltage with the lapse of time;

FIG. 6A is a schematic diagram illustrating reductive decomposition of SOx which is caused in an element portion of the present detection device;

FIG. 6B is a schematic diagram illustrating a reoxidation reaction of sulfur which is caused in the element portion of the present detection device;

FIG. 7 is a graph illustrating a relationship between the application voltage and the output voltage for each air-fuel ratio of an air-fuel mixture in a combustion chamber of the present detection device;

FIG. 8 is a graph illustrating a relationship between the application voltage and the output voltage for each temperature of the element portion of the present detection device;

FIG. 9 is a schematic diagram illustrating a map which is used to determine an upper limit voltage based on an air-fuel ratio of an air-fuel mixture in the combustion chamber of the present detection device and a temperature of the element portion of the present detection device;

FIG. 10 is a table illustrating an example of a combination of an air-fuel ratio of an air-fuel mixture in the combustion chamber of the present internal combustion engine, a temperature of the element portion of the present detection device, and an upper limit voltage;

FIG. 11 is a flowchart illustrating an element temperature control processing routine which is performed by the present detection device;

FIG. 12 is a flowchart illustrating an A/F detection processing routine which is performed by the present detection device; and

FIG. 13 is a flowchart illustrating a SOx concentration determination processing routine which is performed by the present detection device.

### DETAILED DESCRIPTION OF EMBODIMENTS

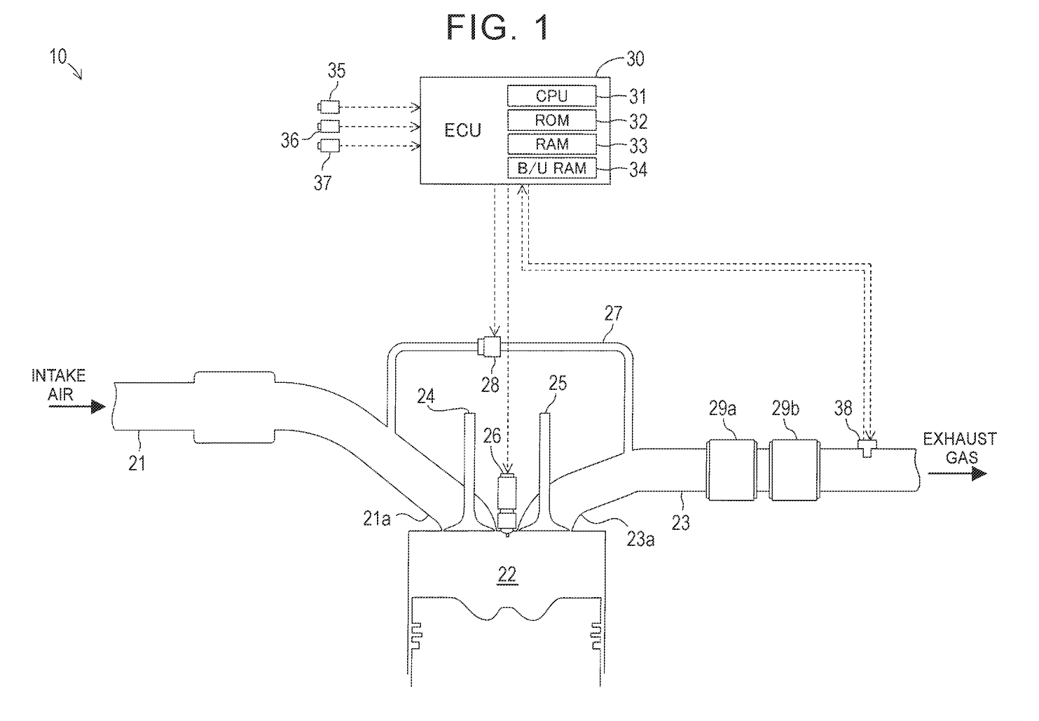

Hereinafter, a gas detection device according to an embodiment of the disclosure (hereinafter also referred to as the “present detection device”) will be described with reference to the accompanying drawings. The present detection device is applied to an internal combustion engine 10 illustrated in FIG. 1. The internal combustion engine 10 is mounted in a vehicle which is not illustrated.

The internal combustion engine 10 is a diesel engine. The internal combustion engine 10 includes an intake air passage 21 including an intake port 21a, a combustion chamber 22, an exhaust gas passage 23 including an exhaust port 23a, an intake valve 24, an exhaust valve 25, a fuel injection valve 26, an exhaust gas recirculation pipe 27, and an EGR control valve 28.

The intake valve 24 is disposed in a cylinder head portion and is driven by an intake cam shaft which is not illustrated to open or close a “communicating portion between the intake port 21a and the combustion chamber 22.” The exhaust valve 25 is disposed in the cylinder head portion and is driven by an exhaust cam shaft which is not illustrated to open or close a “communicating portion between the exhaust port 23a and the combustion chamber 22.”

The fuel injection valve 26 is disposed in the cylinder head portion to inject fuel into the combustion chamber 22. The fuel injection valve 26 directly injects fuel into the combustion chamber 22 in response to an instruction from an ECU 30 which will be described later.

The exhaust gas recirculation pipe 27 and the EGR control valve 28 constitute an EGR device. The exhaust gas recirculation pipe 27 recirculates a part of an exhaust gas flowing in the exhaust gas passage 23 as EGR gas to the intake air passage 21. The EGR control valve 28 controls an amount of EGR gas flowing in the exhaust gas recirculation pipe 27 in response to an instruction from the ECU 30.

A diesel oxidation catalyst (DOC) 29a and a diesel particulate filter (DPF) 29b are inserted into the exhaust gas passage 23.

The DOC 29a is an exhaust gas purification catalyst. The DOC 29a oxidizes unburned components (specifically, HC and CO) in the exhaust gas to purify the exhaust gas using precious metals such as platinum and palladium as a catalyst. That is, by the DOC 29a, HC is oxidized to water and CO₂ and CO is oxidized to CO₂.

The DPF 29b is disposed downstream from the DOC 29a in the exhaust gas passage 23. The DPF 29b is a filter that captures particulates in the exhaust gas. The DPF 29b includes a plurality of passages formed of a porous material (for example, a diaphragm formed of cordierite which is a kind of ceramic). The DPF 29b captures particulates contained in the exhaust gas passing through the diaphragm on pore surfaces of the diaphragm.

An electronic control unit (ECU) 30 includes a CPU 31, a ROM 32, a RAM 33, and a backup RAM 34. The CPU 31 performs reading of data, calculation of numerical values, and outputting of calculation results by sequentially executing a predetermined program (routine). The ROM 32 stores programs which are executed by the CPU, a lookup table (a map), and the like. The RAM 33 temporarily stores data. The backup RAM 34 can store data regardless of whether an ignition key switch (not illustrated) of a vehicle in which the internal combustion engine 10 is mounted is in an ON state or an OFF state.

The ECU 30 is connected to various actuators of the internal combustion engine 10 (such as the fuel injection valve 26 and the EGR control valve 28). The ECU 30 sends out a drive (instruction) signal to the actuators to control the internal combustion engine 10. The ECU 30 is connected to a crank angle sensor 35, an accelerator pedal depression sensor 36, a coolant temperature sensor 37, and a gas sensor 38.

The crank angle sensor 35 generates a signal corresponding to a rotational position of a crank shaft of the internal combustion engine 10 which is not illustrated. The ECU 30 calculates an engine rotation speed NE of the internal combustion engine 10 based on a signal from the crank angle sensor 35.

The accelerator pedal depression sensor 36 detects an amount of operation of an accelerator pedal (not illustrated) (an accelerator depression amount) of the vehicle in which the internal combustion engine 10 is mounted and outputs a signal indicating an accelerator pedal operation amount Ap. The coolant temperature sensor 37 detects a temperature of a coolant (a coolant temperature THW) for cooling the internal combustion engine 10 and outputs a signal indicating the coolant temperature THW.

The gas sensor 38 is a one-cell limit current type gas sensor and is disposed in the exhaust gas passage 23. The gas sensor 38 is disposed downstream from the DOC 29a and the DPF 29b in the exhaust gas passage 23.

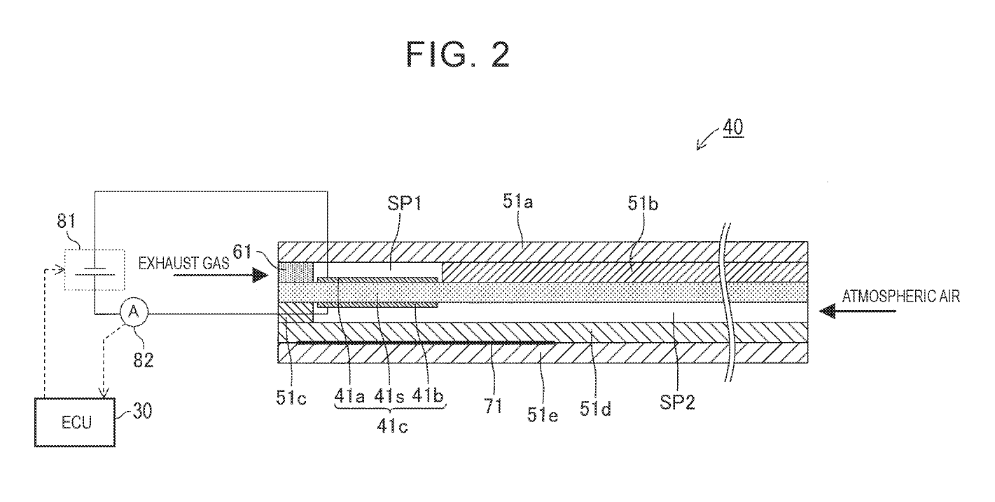

(Configuration of gas sensor) A configuration of the gas sensor 38 will be described below with reference to FIG. 2. An element portion 40 of the gas sensor 38 includes a solid electrolyte 41s, a first alumina layer 51a, a second alumina layer 51b, a third alumina layer 51c, a fourth alumina layer 51d, a fifth alumina layer 51e, a diffusion resistor portion (a diffusion rate control layer) 61, and a heater 71. A first electrode 41a and a second electrode 41b are fixed to the solid electrolyte 41s.

The solid electrolyte 41s is a thin plate member with oxide ion conductivity which contains zirconia. Zirconia forming the solid electrolyte 41s may contain an element such as scandium (Sc) and yttrium (Y).

Each of the first alumina layer 51a to the fifth alumina layer 51e is a dense (gas-non-transmitting) layer (a dense thin plate member) containing alumina. The diffusion resistor portion 61 is a porous diffusion rate control layer and is a gas-transmitting layer (a thin plate member).

The heater 71 is a thin plate member of cermet containing platinum (Pt) and ceramics (such as alumina) and is a heat emitter that emits heat with supply of electric power. The heater 71 is connected to a DC power source (not illustrated) via a “power supply line (not illustrated) which is switched between a power supplied state and a power non-supplied state by the ECU 30.” An amount of heat emitted from the heater 71 can be changed by causing the ECU 30 to control a duty ratio Rd which is a ratio of a “time in the power supplied state” to a “sum of the time in the power supplied state and a time in the power non-supplied state.”

The layers of the element portion 40 are stacked from the bottom in the order of the fifth alumina layer 51e, the fourth alumina layer 51d, the third alumina layer 51c, the solid electrolyte 41s, the diffusion resistor portion 61, the second alumina layer 51b, and the first alumina layer 51a. An internal space SP1 and an air introduction passage SP2 are formed in the element portion 40.

The internal space SP1 is formed by the first alumina layer 51a, the solid electrolyte 41s, the diffusion resistor portion 61, and the second alumina layer 51b. The “exhaust gas of the internal combustion engine 10 as a sample gas” is introduced into the internal space SP1 from the exhaust gas passage 23 via the diffusion resistor portion 61. That is, the internal space SP1 communicates with the inside of the exhaust gas passage 23 via the diffusion resistor portion 61.

The air introduction passage SP2 is formed by the solid electrolyte 41s, the third alumina layer 51c, and the fourth alumina layer 51d. The air introduction passage SP2 is open to atmospheric air outside the internal combustion engine 10.

The first electrode 41a is fixed to one surface of the solid electrolyte 41s (specifically, the surface of the solid electrolyte 41s defining the internal space SP1). The first electrode 41a is a negative electrode. The first electrode 41a is a porous cermet electrode containing platinum (Pt) as a main component.

The second electrode 41b is fixed to the other surface of the solid electrolyte 41s (specifically, the surface of the solid electrolyte 41s defining the air introduction passage SP2). The second electrode 41b is a positive electrode. The second electrode 41b is a porous cermet electrode containing platinum (Pt) as a main component.

The first electrode 41a and the second electrode 41b are disposed to face each other with the solid electrolyte 41s interposed therebetween. That is, the first electrode 41a, the second electrode 41b, and the solid electrolyte 41s constitute an electrochemical cell 41c having an oxygen discharge capability based on an oxygen pumping effect which will be described later. The electrochemical cell 41c is heated by the heater 71 such that an element temperature of the electrochemical cell 41c (a sensor element temperature) Tcl is higher than an activation temperature Tat which will be described later.

The layers including the solid electrolyte 41s and the first to fifth alumina layers 51a to 51e are formed in a sheet shape, for example, using a doctor blade method or an extrusion molding method. The first electrode 41a, the second electrode 41b, and wires for supplying power to the electrodes, and the like are formed, for example, using a screen printing method. By stacking and baking such sheets as described above, the element portion 40 having the above-mentioned structure is integrally formed.

A material of the first electrode 41a is not limited to the above-mentioned materials, and can be selected, for example, from materials containing an platinum group metal such as platinum (Pt), rhodium (Rh), or palladium (Pd) or an alloy thereof as a main component. The material of the first electrode 41a is not particularly limited as long as SOx contained in the sample gas guided into the internal space SP1 via the diffusion resistor portion 61 can be reductively decomposed when a decomposition start voltage (specifically, a voltage of about 0.6 V or more)) of SOx which will be described later is applied between the first electrode 41a and the second electrode 41b. The decomposition start voltage of SOx is also referred to as a “specific voltage” for the purpose of convenience.

The gas sensor 38 includes a power supply circuit 81 and an ammeter 82. The power supply circuit 81 and the ammeter 82 are connected to the ECU 30.

The power supply circuit 81 is configured to apply an application voltage Vm between the first electrode 41a and the second electrode 41b. The application voltage Vm has a positive value when the potential of the second electrode 41b is higher than the potential of the first electrode 41a. The power supply circuit 81 can change the application voltage Vm under control of the ECU 30.

The ammeter 82 measures a magnitude of an output current (an electrode current) Im which is a current flowing between the first electrode 41a and the second electrode 41b (accordingly, a current flowing in the solid electrolyte 41s) and outputs the measured value to the ECU 30. The output current Im has a positive value when a current flows from the second electrode 41b to the first electrode 41a via the solid electrolyte 41s.

<Outline of operation> An operation of the present detection device will be described below. The present detection device detects an oxygen concentration of the exhaust gas (the sample gas) discharged from the internal combustion engine 10. The present detection device detects an air-fuel ratio (A/F) of an air-fuel mixture in the combustion chamber 22 based on the oxygen concentration in the air-fuel mixture. The air-fuel ratio of the air-fuel mixture in the combustion chamber 22 is also referred to as an “air-fuel ratio A/F of an engine.” The present detection device determines whether a SOx concentration in the sample gas is higher than a predetermined value (determination of whether the SOx concentration is higher than a predetermined value is also referred to as “determination of a SOx concentration” for the purpose of convenience).

The determination of a SOx concentration requires a certain time (specifically several seconds). When the air-fuel ratio A/F of the engine changes during the determination of the SOx concentration, the SOx concentration of the sample gas also changes. Therefore, the present detection device determines the SOx concentration when a change of the air-fuel ratio A/F of the engine with respect to time is small.

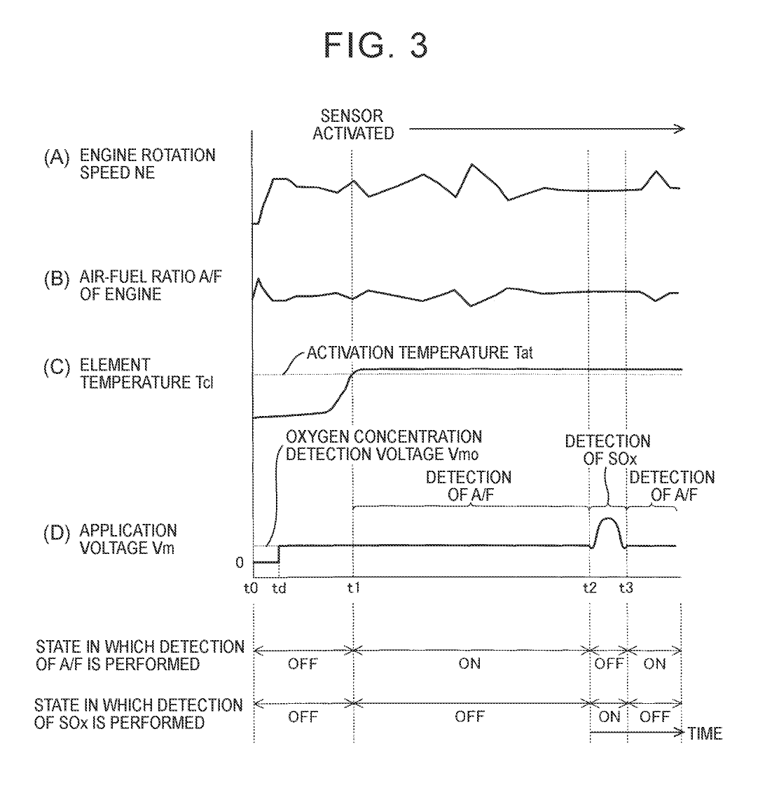

An example of changes of various parameters when the internal combustion engine 10 is started, then the present detection device starts detection of the air-fuel ratio A/F of the engine, and then determines the SOx concentration at an appropriate time is illustrated in the timing chart of FIG. 3.

In the example illustrated in (A) of FIG. 3, at time t0 which is a time point at which the operation of the internal combustion engine 10 is started, the ECU 30 controls the duty ratio Rd such that the heater 71 emits heat to heat the electrochemical cell 41c. As a result, the element temperature Tcl of the electrochemical cell 41c increases. Thereafter, at time t1, the element temperature Tcl is higher than an “activation temperature Tat which is a temperature at which the electrochemical cell 41c exhibits oxide ion conductivity.”

When the element temperature Tcl is higher than the activation temperature Tat and the gas sensor 38 enters a sensor activated state, the ECU 30 starts detection of the oxygen concentration and detection (acquisition) of the air-fuel ratio A/F of the engine based on the detected oxygen concentration.

At time td which is a time point between time t0 and time t1, the ECU 30 sets the application voltage Vm to an oxygen concentration detection voltage Vmo. In this example, the oxygen concentration detection voltage Vmo is 0.3 V. That is, the first electrode 41a serves as a negative electrode and the second electrode 41b serves as a positive electrode. Since the oxygen concentration detection voltage Vmo is higher than a voltage at which decomposition of oxygen (O₂) in the first electrode 41a occurs (a decomposition start voltage of oxygen), oxygen contained in the exhaust gas is reductively 26 decomposed into oxide ions (O²⁻) in the first electrode 41a. On the other hand, the oxygen concentration detection voltage Vmo is lower than a voltage at which decomposition of oxygen-containing components other than oxygen occurs.

Oxide ions which have been reductively decomposed in the first electrode 41a are conducted to the second electrode 41b via the solid electrolyte 41s and are changed to oxygen (O₂) in the second electrode 41b. Oxygen is discharged to the atmospheric air via the air introduction passage SP2. Migration of oxide ions from the negative electrode (the first electrode 41a) to the positive electrode (the second electrode 41b) via the solid electrolyte 41s is also referred to as an “oxygen pumping effect.”

By the migration of oxide ions due to the oxygen pumping effect, a current (that is, an output current Im) is generated between the first electrode 41a and the second electrode 41b. In general, when the application voltage Vm increases, an amount of oxygen which is reductively decomposed in the first electrode 41a increases and thus the output current Im increases. However, since an amount of exhaust gas reaching the first electrode 41a (accordingly, an amount of oxygen contained in the exhaust gas) is limited by the diffusion resistor portion 61, the output current Im does not increase even if the application voltage Vm is higher than a certain voltage. This state is also referred to as a “diffusion rate control state.” In other words, the diffusion rate control state is a state in which the capability of reductively decomposing oxygen in the first electrode 41a is greater than the capability of supplying oxygen to the first electrode 41a.

The output current Im in the diffusion rate control state is also referred to as a “limit current.” The limit current increases as an amount of oxygen supplied to the first electrode 41a increases. The amount of supplied to the first electrode 41a increases as the oxygen concentration in the exhaust gas increases. Accordingly, the limit current and the oxygen concentration in the exhaust gas have one-to-one correspondence.

Therefore, the ECU 30 acquires the oxygen concentration in the exhaust gas by applying the value of the limit current to a “map indicating a relationship between the value of the limit current and the oxygen concentration in the exhaust gas” stored in the ROM 32. The air-fuel ratio A/F of the engine increases as the oxygen concentration in the exhaust gas increases. Therefore, the ECU 30 acquires the air-fuel ratio A/F of the engine by applying the oxygen concentration in the exhaust gas to a “map indicating a relationship between the oxygen concentration in the exhaust gas and the air-fuel ratio A/F of the engine” stored in the ROM 32.

The ECU 30 may directly acquire the air-fuel ratio A/F of the engine by applying the value of the limit current to a “map indicating a relationship between the value of the limit current and the air-fuel ratio A/F of the engine” stored in the ROM 32.

(Principle of detection of SOx concentration) A method of determining the SOx concentration in the exhaust gas (the sample gas) will be described below. The oxygen pumping effect occurs for “oxygen-containing components such as SOx (sulfur oxide) and H₂O (water)” containing an oxygen atom in a molecule thereof. Specifically, when the application voltage Vm is higher than a decomposition start voltage of any one of the oxygen-containing components, the corresponding oxygen-containing component is reductively decomposed to produce oxide ions. The oxide ions produced by the reductive decomposition are conducted from the first electrode 41a to the second electrode 41b by the oxygen pumping effect. In other words, the output current Im increases due to the reductive decomposition of the oxygen-containing component.

However, since the SOx concentration in the exhaust gas is generally very low, the current resulting from the reductive decomposition of SOx (that is, an increase of the output current Im) is very small. Accordingly, it is difficult to accurately extract an increase of the output current Im (a part of the output current Im contributing to reductive decomposition of SOx) resulting from the reductive decomposition of SOx by only increasing the application voltage Vm to be higher than the decomposition start voltage of SOx (about 0/6 V).

Therefore, through hard study, the inventor of the disclosure obtained knowledge that the SOx concentration in the exhaust gas can be accurately detected by performing a “step-up process” of gradually stepping up the application voltage Vm from a first voltage lower than the decomposition start voltage of SOx to a second voltage higher than the decomposition start voltage of SOx and then performing a “step-down process” of gradually stepping down the application voltage Vm to a third voltage lower than the decomposition start voltage of SOx. The first voltage and the third voltage in this embodiment are equal. The first voltage and the third voltage are hereinafter also referred to as a “lower limit voltage Vbt.” On the other hand, the second voltage is hereinafter also referred to as an “upper limit voltage Vup.”

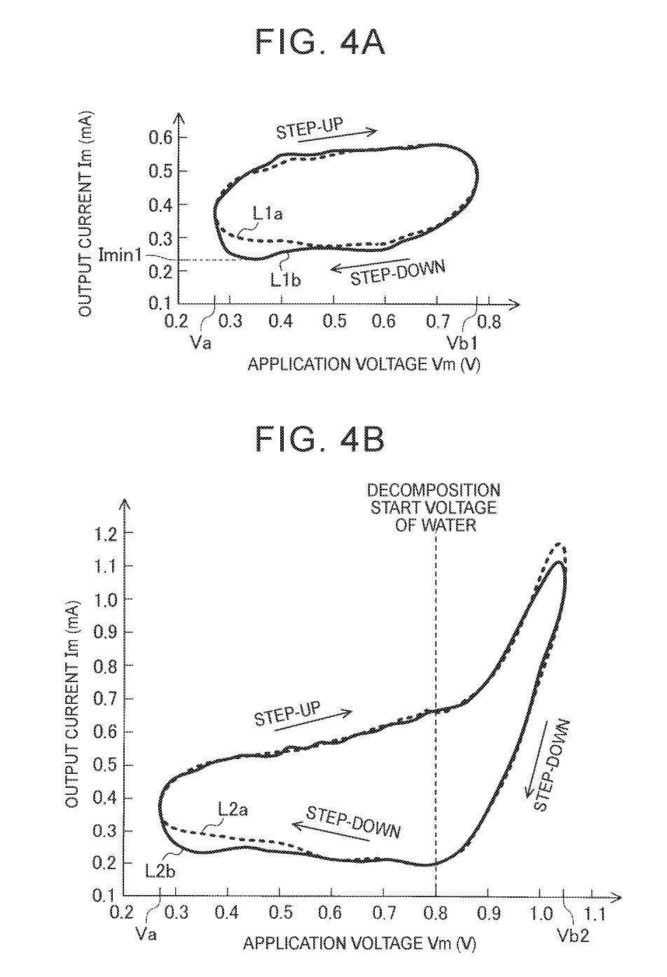

An example of a change of the output current Im when the step-up process and the step-down process are performed is illustrated in FIG. 4A. In FIG. 4A, a change of the output current Im when the exhaust gas does not contain SOx is indicated by a dotted line L1a, and a change of the output current Im when the exhaust gas contains SOx is indicated by a solid line L1b. In the example illustrated in FIG. 4A, the lower limit voltage Vbt is a voltage Va (about 0.27 V), and the upper limit voltage Vup is a voltage Vb1 (about 0.78 V).

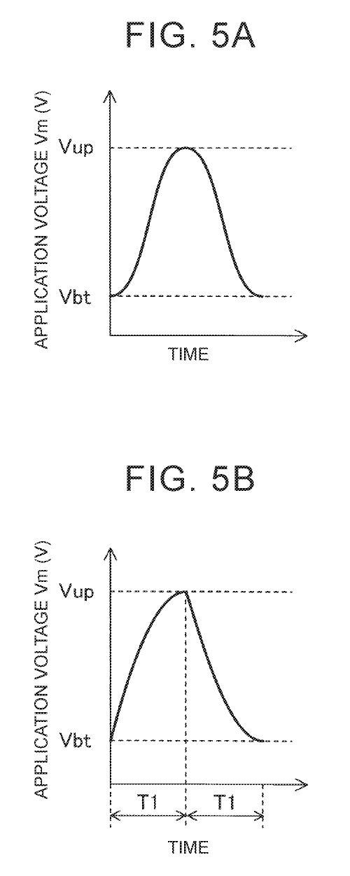

A waveform of the application voltage Vm (the change of the application voltage Vm with the lapse of time) when the changes of the output current Im indicated by the dotted line L1a and the solid line L1b in FIG. 4A occur is illustrated in FIG. 5A. As can be seen from FIG. 5A, the waveform of the application voltage Vm in the step-up process and the step-down process is one period of a sinusoidal wave. In this example, the period of the waveform (sinusoidal wave) of the application voltage Vm is a value included from 0.5 Hz to 5 Hz.

The waveform of the application voltage Vm in the step-up process and the step-down process may be a waveform illustrated in FIG. 5B, that is, the waveform of the application voltage Vm in the step-up process may be similar to a waveform of an inter-terminal voltage of a capacitor when the capacitor is charged, and the waveform of the application voltage Vm in the step-down process may be similarly to a waveform of an inter-terminal voltage of a capacitor when the capacitor is discharged. In this case, times TI which are required for the step-up process and the step-down process are values included from 100 msec to 10 sec.

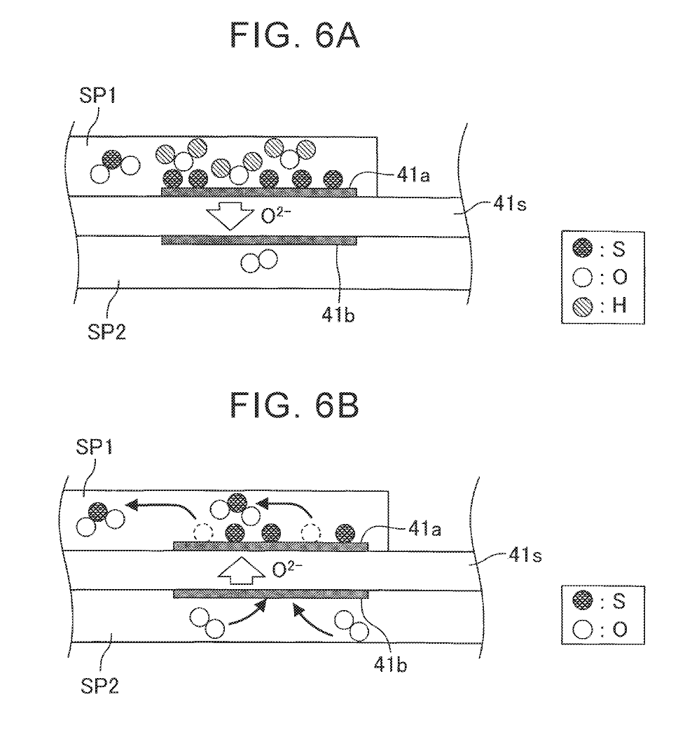

When the application voltage Vm is higher than the decomposition start voltage of SOx during the step-up process, SOx in the exhaust gas is reductively decomposed into S and O²⁻ in the first electrode 41a (the negative electrode) as illustrated in FIG. 6A. As a result, a product of the reductive decomposition of SOx (that is, sulfur (S)) is adsorbed (deposited) in the first electrode 41a.

Thereafter, when the application voltage Vm is lower than the decomposition start voltage of SOx during the step-down process, a reaction in which “S adsorbed on the first electrode 41a” and O²⁻ are combined to produce SOx (hereinafter also referred to as a “reoxidation reaction of S”) occurs as illustrated in FIG. 6B. At this time, the output current Im changes (decreases) due to the reoxidation reaction of S. Hereinafter, the “change of the output current Im due to the reoxidation reaction of S” is also referred to as a “reoxidation current change.”

Specifically, when the exhaust gas contains SOx (that is, the solid line L1b in FIG. 4A) and the application voltage Vm in the step-down process is lower than the decomposition start voltage of SOx, a reaction rate of the reoxidation reaction of S (the number of sulfur molecules which are re-oxidized per unit time) increases temporarily. Thereafter, the reaction rate of the reoxidation reaction of S decreases with a decrease in S adsorbed on the first electrode 41a. As a result, as can be seen from the solid line L1b, the output current Im changes to the current minimum value Imin1.

On the other hand, when the exhaust gas does not contain SOx (that is, the dotted line L1a), a temporary decrease of the output current Im does not occur even if the application voltage Vm in the step-down process is lower than the decomposition start voltage of SOx. Accordingly, the SOx concentration can be determined based on a correlation between the SOx concentration in the exhaust gas and the minimum value of the output current Im in the step-down process (that is, the reoxidation current change). A specific method of determining the SOx concentration will be described later.

When the exhaust gas contains SOx, the reoxidation current change occurs in the step-down process, and a sufficient amount of S corresponding to the SOx concentration needs to be adsorbed (deposited) on the first electrode 41a when the application voltage Vm is higher than the decomposition start voltage of SOx. However, when the application voltage Vm is higher than the decomposition start voltage of water, water is reductively decomposed in the first electrode 41a.

An example of a change of the output current Im when the upper limit voltage Vup is higher than the decomposition start voltage of water (about 0.8 V) is illustrated in FIG. 4B. In FIG. 4B, the change of the output current Im when the exhaust gas does not contain SOx is indicated by a dotted line L2a, and the change of the output current Im when the exhaust gas contains SOx is indicated by a solid line L2b. In the example illustrated in FIG. 4B, the lower limit voltage Vbt is a voltage Va and the upper limit voltage Vup is a voltage Vb2 (about 1.03 V).

The water concentration in the exhaust gas is much higher than the SOx concentration in the exhaust gas. Accordingly, as can be seen from the dotted line L2a and the solid line L2b, when the application voltage Vm increases in a range which is higher than the decomposition start voltage of water (about 0.8 V) in the step-up process, the output current Im increases rapidly. Thereafter, when the application voltage Vm decreases in a range which is higher than the decomposition start voltage of water in the step-down process, the output current Im decreases rapidly. The voltage at which the output current Im starts rapid increase in the step-up process is hereinafter also referred to as an “decomposition activation voltage of water.”

With a rapid change of the output current Im due to the reductive decomposition of water, there is a likelihood that the “reoxidation current change due to the reoxidation reaction of S” will be affected. More specifically, when the application voltage Vm is higher than the decomposition start voltage of water, the decomposition start voltage of water is higher than the decomposition start voltage of SOx and thus both SOx and water are reductively decomposed. As described above, the water concentration in the exhaust gas is much higher than the SOx concentration in the exhaust gas. Accordingly, when the application voltage Vm is higher than the decomposition activation voltage of water, the number of water molecules which is reductively decomposed per unit time (the reductive decomposition rate of water) is much larger than the number of SOx molecules which is reductively decomposed per unit time (the reductive decomposition rate of SOx). Accordingly, there is a likelihood that the output current Im in the reoxidation reaction of S occurring in the step-down process will change more in comparison with a case in which reductive decomposition of water does not occur due to the rapid increase of the output current Im resulting from the reductive decomposition of water when the application voltage Vm is higher than the decomposition activation voltage of water and the significant decrease of the output current Im occurring when the application voltage Vm is lower than the decomposition activation voltage of water.

The reductive decomposition rate of water increases as the water concentration in the exhaust gas increases. Accordingly, when the application voltage Vm is higher than the decomposition activation voltage of water, there is a likelihood that the reductive decomposition rate of SOx will change with the water concentration in the exhaust gas. That is, there is a likelihood that an amount of S adsorbed on the first electrode 41a will change by a factor (specifically the water concentration in the exhaust gas) other than the SOx concentration in the exhaust gas.

Conclusively, in order to accurately determine the SOx concentration in the exhaust gas, the upper limit voltage Vup needs to be set to a value higher a certain degree than the decomposition start voltage of SOx such that a sufficient amount of S corresponding to the SOx concentration in the exhaust gas is adsorbed on the first electrode 41a. On the other hand, when the upper limit voltage Vup is higher than the decomposition activation voltage of water, a rapid change of the output current Im due to the reductive decomposition of water occurs, and thus there is a likelihood that the reoxidation current change will be affected and determination accuracy of the SOx concentration will deteriorate. Therefore, the ECU 30 sets the upper limit voltage Vup to a value slightly smaller than the decomposition activation voltage of water.

The decomposition activation voltage of water changes with the air-fuel ratio A/F of the engine and the element temperature Tcl of the electrochemical cell 41c. First, a relationship between the air-fuel ratio A/F of the engine and the decomposition activation voltage of water will be described below.

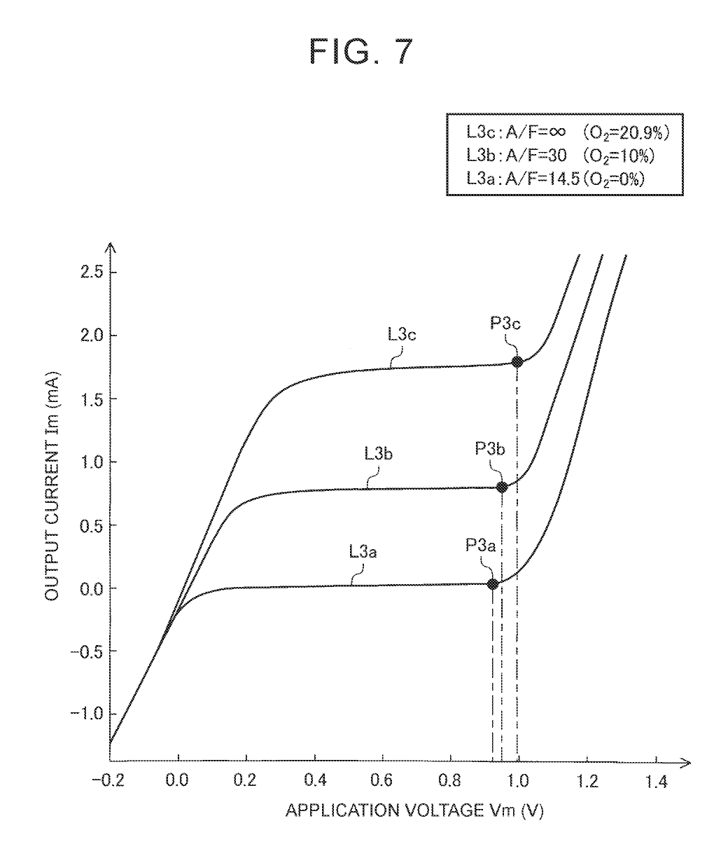

The change of the output current Im for each air-fuel ratio A/F of the engine when the exhaust gas does not contain SOx and the application voltage Vm increases is indicated by curves L3a to L3c in FIG. 7. Each of points P3a to P3c in FIG. 7 indicates the decomposition activation voltage of water in the curves L3a to L3c. As can be seen from points P3a to P3c, as the air-fuel ratio A/F of the engine increases, the decomposition activation voltage of water increases.

The reason thereof is as follows. That is, when the air-fuel ratio A/F of the engine decreases, an amount of fuel which is combusted in a combustion stroke of the internal combustion engine 10 increase and thus an amount of water which is produced with the combustion reaction increases. Accordingly, when the air-fuel ratio A/F of the engine decreases, the water concentration in the exhaust gas increases. When the water concentration in the exhaust gas is high, the number of water molecules which are reductively decomposed increases and thus more water molecules are reductively decomposed even when the application voltage Vm is low in comparison with a case in which the water concentration in the exhaust gas is low. Accordingly, when the water concentration in the exhaust gas is high, the application voltage Vm at which the rapid increase of the output current Im decreases in comparison with a case in which the water concentration in the exhaust gas is low. In other words, as the water concentration in the exhaust gas increases (that is, as the air-fuel ratio A/F of the engine decreases), the decomposition activation voltage of water decreases.

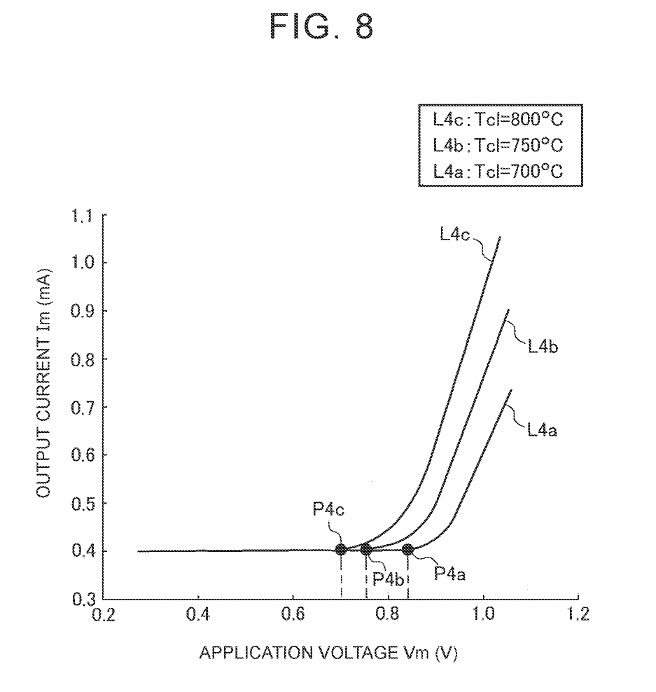

A relationship between the element temperature Tcl and the decomposition activation voltage of water will be described below. The change of the output current Im for each element temperature Tcl when the application voltage Vm increases is indicated by curves L4a to L4c in FIG. 8. Points P4a to P4c in FIG. 8 indicate the decomposition activation voltage of water in the curves 14a to L4c.

As can be seen from the points P4a to P4c, as the element temperature Tcl increases, the decomposition activation voltage of water decreases. This is because as the element temperature Tcl increases, catalyst activity of precious metal contained in the first electrode 41a is more improved.

The ECU 30 estimates (acquires) the element temperature Tcl based on impedance Rc of the electrochemical cell 41c. Specifically, as the element temperature Tcl increases, the impedance Rc decreases. The ECU 30 sets the application voltage Vm to a high frequency with amplitude of 0.4 V in a predetermined period when the impedance Rc is detected. In the waveform of the application voltage Vm illustrated in (D) of FIG. 3, a high-frequency voltage which is applied at the time of detecting the impedance Rc is not illustrated.

The ECU 30 acquires the impedance Rc using a well-known method based on the output current Im when a high-frequency voltage is applied to the electrochemical cell 41c. Then, the ECU 30 acquires the element temperature Tcl by applying the acquired impedance Rc to a “map indicating a relationship between the impedance Rc and the element temperature Tcl” stored in the ROM 32.

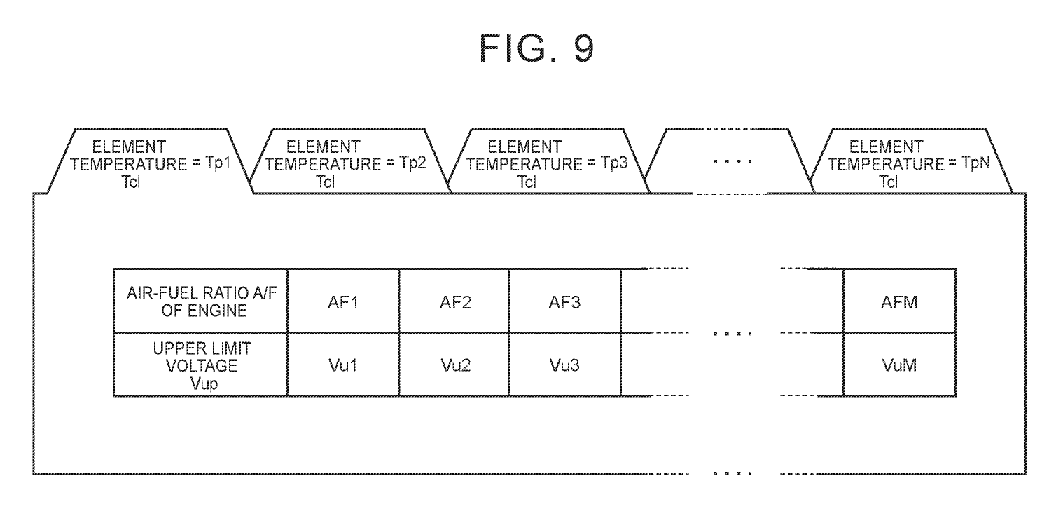

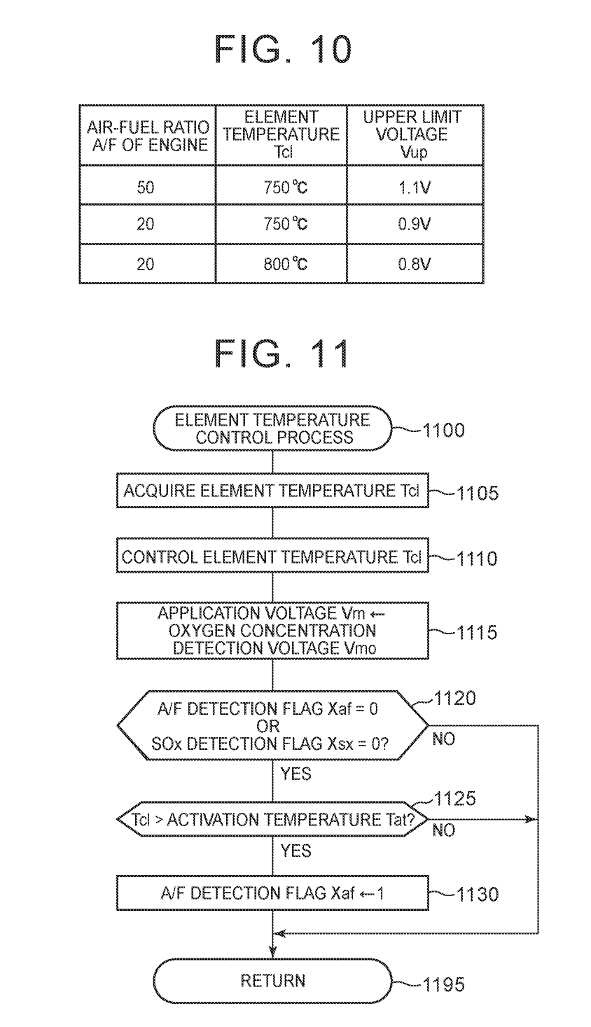

The ECU 30 determines (acquires) the upper limit voltage Vup by applying the “more recent air-fuel ratio A/F of the engine acquired by the gas sensor 38” and the “element temperature Tcl acquired based on the impedance Re” to a “map indicating a relationship between the air-fuel ratio A/F of the engine, the element temperature Tcl, and the upper limit voltage Vup.” Specifically, the ECU 30 sets the upper limit voltage Vup to a larger value as the air-fuel ratio A/F of the engine becomes larger. In addition, the ECU 30 sets the upper limit voltage Vup to a smaller value as the element temperature Tcl becomes higher. This map (lookup table) is schematically illustrated in FIG. 9. An example of a combination of the air-fuel ratio A/F of the engine, the element temperature Tcl, and the upper limit voltage Vup which are acquired from the map illustrated in FIG. 9 is illustrated in FIG. 10.

(Method of determining SOx concentration) As described above, the ECU 30 determines the SOx concentration based on the reoxidation current change due to the reoxidation reaction of S. More specifically, the ECU 30 determines that the SOx concentration is equal to or higher than a predetermined value when a current difference Idf is equal to or greater than a predetermined current threshold value Ith.

The ECU 30 calculates the current difference Idf as a difference between a reference current Ib and a current minimum value Imin (that is, Idf=Ib−Imin). The reference current Ib is the output current Im when the application voltage Vm is equal to the oxygen concentration detection voltage Vmo before the step-up process is started. On the other hand, the current minimum value Imin is a minimum value of the output current Im in a “period in which the application voltage Vm is lower than a current acquisition start voltage Vsem” in the step-down process.

The current acquisition start voltage Vsem is a voltage which is higher than the lower limit voltage Vbt and equal to or lower than the decomposition start voltage of SOx (about 0.6 V as described above). In this example, the current acquisition start voltage Vsem is set to the decomposition start voltage of SOx.

(Specific operation) A specific operation of the ECU 30 associated with control of the element temperature Tcl, acquisition of the air-fuel ratio A/F of the engine, and determination of the SOx concentration in the exhaust gas will be described below. The CPU 31 of the ECU 30 (hereinafter also simply referred to as a “CPU”) performs an “element temperature control processing routine” illustrated in the flowchart of FIG. 11 at predetermined time intervals.

Accordingly, at an appropriate time, the CPU starts the processing routine from Step 1100 in FIG. 11, sequentially performs the processes of Steps 1105 to 1115, and then performs the process of Step S1120.

Step 1105: The CPU acquires the element temperature Tcl of the electrochemical cell 41c based on the impedance Rc of the electrochemical cell 41c through the above-mentioned process. Step S1110: The CPU sets the duty ratio Rd such that the element temperature Tcl approaches a target temperature Ttg. Specifically, when the element temperature Tcl is lower than the target temperature Ttg, the CPU increases the value of the duty ratio Rd. On the other hand, when the element temperature Tcl is higher than the target temperature Ttg, the CPU decreases the value of the duty ratio Rd.

Step 1115: The CPU sets the application voltage Vm to the oxygen concentration detection voltage Vmo. The above-mentioned time Td (that is, the time at which the application voltage Vm is first set to the oxygen concentration detection voltage Vmo) is a time at which this step is first performed after the internal combustion engine 10 is started (that is, after a time at which an ignition key switch of a vehicle in which the internal combustion engine 10 is mounted is changed from an OFF position to an ON position).

In Step 1120, the CPU determines whether the value of an A/F detection flag Xaf is “0” and the value of a SOx determination flag Xsx is “0.” The values of the A/F detection flag Xaf and the SOx determination flag Xsx are set to “0” in an initial routine (not illustrated) which is performed by the CPU at the time of starting of the internal combustion engine 10.

When the internal combustion engine 10 is just started, the values of the A/F detection flag Xaf and the SOx determination flag Xsx are “0.” In this case, the CPU determines “YES” in Step 1120 and determines whether the element temperature Tcl is higher than the activation temperature Tat in Step 1125.

When the element temperature Tcl is higher than the activation temperature Tat (that is, when the gas sensor 38 is in a sensor-activated state), the CPU determines “YES” in Step 1125 and sets the value of the A/F detection flag Xaf to “1” in Step 1130. Subsequently, the CPU ends this routine in Step S1195.

On the other hand, when the element temperature Tcl is equal to or lower than the activation temperature Tat, the CPU determines “NO” in Step 1125 and then directly performs Step S1195.

When the determination condition in Step 1120 is not satisfied (that is, when the value of the A/F detection flag Xaf and/or the SOx determination flag Xsx is “1”), the CPU determines “NO” in Step 1120 and then directly performs Step 1195.

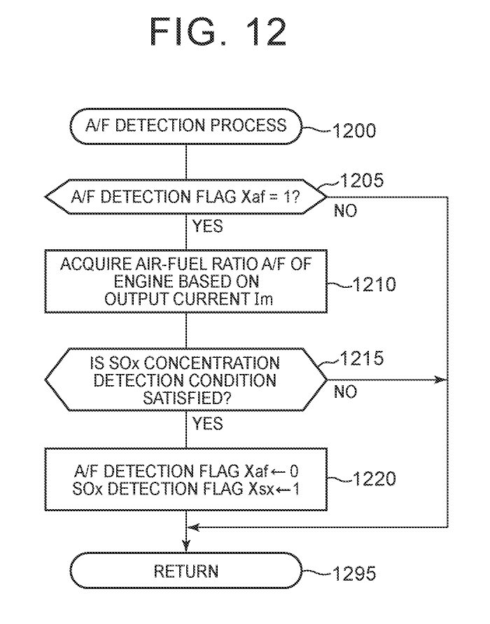

A specific operation of the ECU 30 associated with acquisition of the air-fuel ratio A/F of the engine will be described below. The CPU repeatedly performs an “A/F detection processing routine” illustrated in the flowchart of FIG. 12 at predetermined time intervals.

Accordingly, at an appropriate time, the CPU starts the processing routine from Step 1200 in FIG. 12 and determines whether the value of the A/F detection flag Xaf is “1” in Step S1205.

When the value of the A/F detection flag Xaf is “0,” the CPU determines “NO” in Step 1205 and ends this routine in Step 1295.

On the other hand, when the value of the A/F detection flag Xaf is “1,” the CPU determines “YES” in Step 1205 and acquires the air-fuel ratio A/F of the engine based on the output current Im in Step 1210. That is, the CPU acquires the oxygen concentration in the exhaust gas based on the output current Im when the application voltage Vm is the oxygen concentration detection voltage Vmo and acquires the air-fuel ratio A/F of the engine based on the acquired oxygen concentration as described above. The CPU stores the acquired air-fuel ratio A/F of the engine in the RAM 33.

Subsequently, in Step 1215, the CPU determines whether a SOx concentration detection condition is satisfied. The SOx concentration detection condition is a condition which is satisfied when all the following conditions of (a) to (d) are satisfied.

(a) The internal combustion engine 10 is in a warmed-up state (specifically, the coolant temperature THW is higher than a predetermined warm-up temperature Tth). (b) The gas sensor 38 is in the sensor-activated state. (c) The internal combustion engine 10 is not in a fuel-cut state. (d) The air-fuel ratio A/F of the engine is stabilized. Specifically, the operating state of the internal combustion engine 10 is an idling state or a operating state of the vehicle in which the internal combustion engine 10 is mounted is a normal driving state. Whether the operating state of the internal combustion engine 10 is an idling state is determined by determining whether a “state in which the accelerator pedal operation amount Ap is “0” and the engine rotation speed NE is higher than a predetermined rotation speed” is continuously maintained for an “idling state determination time” or more. Whether the operating state of the vehicle is the normal driving state is determined by determining whether a “state in which a change per unit time of the accelerator pedal operation amount Ap is less than a predetermined threshold change and a change per unit time of the speed of the vehicle in which the internal combustion engine 10 is mounted and which is detected by a vehicle speed sensor (not illustrated) is less than a predetermined threshold acceleration” is continuously maintained for a “normal driving determination time” or more.

The following condition of (e) may be added to the conditions constituting the SOx concentration detection condition. (e) Determination of the SOx concentration is not performed from the start time of the internal combustion engine 10 to the current time.

When the SOx concentration detection condition is satisfied, the CPU determines “YES” in Step 1215, and sets the value of the A/F detection flag Xaf to “0” and sets the value of the SOx determination flag Xsx to “1” in Step 1220. Subsequently, the CPU performs Step 1295.

When the determination condition in Step 1215 is not satisfied (that is, when the SOx concentration detection condition is not satisfied), the CPU determines “NO” in Step 1215 and then directly performs Step 1295.

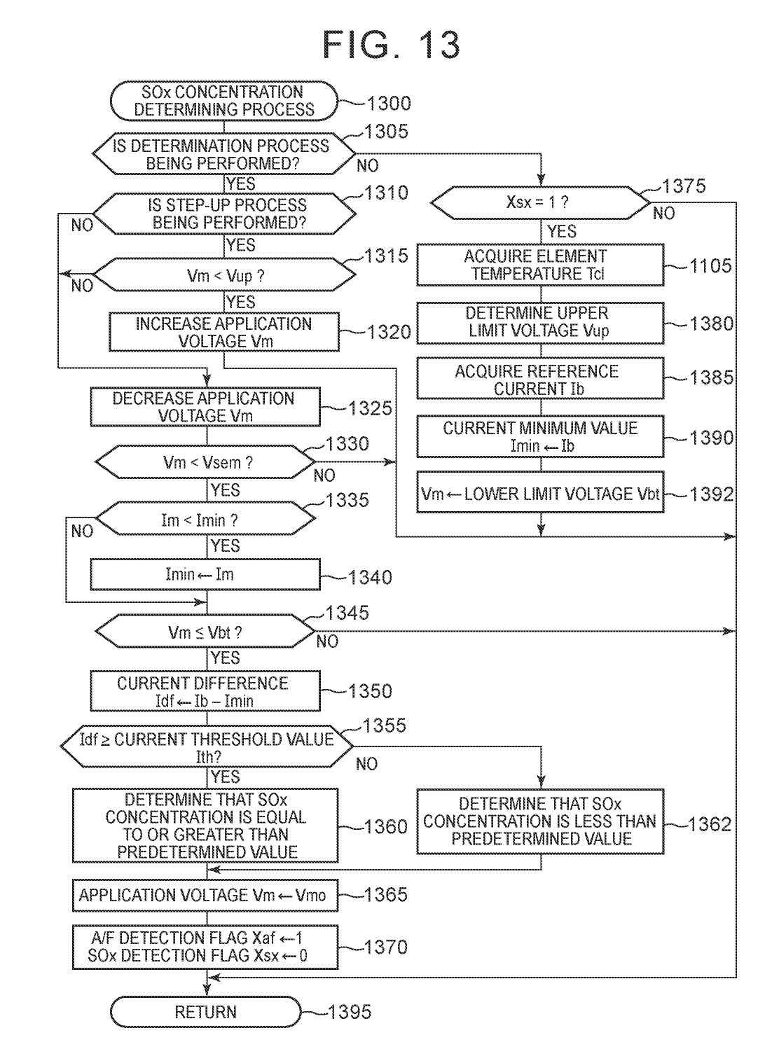

A specific operation of the ECU 30 associated with determination of the SOx concentration will be described below. The CPU repeatedly performs an “SOx concentration determination processing routine” illustrated in the flowchart of FIG. 13 at predetermined time intervals. In the flowchart illustrated in FIG. 13, the same steps as the steps illustrated in the flowchart of FIG. 11 are referenced by the same step numbers as in FIG. 11.

Accordingly, at an appropriate time, the CPU starts the processing routine from Step 1300 in FIG. 13, and determines whether the SOx concentration determination process (specifically, one of the step-up process and the step-down process) has been already performed in Step 1305.

(A) When the value of the SOx determination flag Xsx is “0,” it is assumed that the SOx concentration determination process is not performed and the value of the SOx determination flag Xsx is “0” (for example, immediately after the internal combustion engine 10 is started). In this case, the CPU determines “NO” in Step 1305, and determines whether the value of the SOx determination flag Xsx is “1” in Step 1375. On the above-mentioned assumption, since the value of the SOx determination flag Xsx is “0,” the CPU determines “NO” in Step 1375 and then directly ends this routine in Step 1395.

(B) Immediately after the value of the SOx determination flag Xsx is changed to “1,” it is assumed that this routine is first performed after the value of the SOx determination flag Xsx is changed from “0” to “1.” In this case, when the CPU has performed Steps 1305 to 1375, the CPU determines “YES” in Step 1375 and then performs Step 1105. After the element temperature Tcl is acquired in Step 1105, the CPU sequentially performs the processes of Steps 1380 to 1892 and then performs Step 1395.

Step 1380: The CPU acquires the upper limit voltage Vup by applying the air-fuel ratio A/F of the engine acquired immediately before the value of the SOx determination flag Xsx is finally changed from “0” to “1” in the routine of FIG. 12 and the element temperature Tcl acquired in Step 1105 of FIG. 13 to the map illustrated in FIG. 9. Step 1385: The CPU acquires the “output current Im when the application voltage Vm before the step-up process is started is the oxygen concentration detection voltage Vmo” as a reference current Ib.

Step 1390: The CPU sets the value of the current minimum value Imin as the reference current Ib. Step 1392: The CPU sets the application voltage Vm to the lower limit voltage Vbt (0.27 V in this example). That is, the step-up process is started at this time point and then the application voltage Vm increases gradually.

(C) When this routine is secondly performed after the value of the SOx determination flag Xsx is changed to “1,” it is assumed that this routine is secondly performed after the value of the SOx determination flag Xsx is changed to “1” (that is, this routine is firstly performed after the step-up process is started). In this case, the CPU determines “YES” in Step 1305 and determines whether the step-up process is being performed in Step 1310.

Since the step-up process is being performed on the above-mentioned assumption, the CPU determines “YES” in Step 1310, and determines whether the application voltage Vm is less than the upper limit voltage Vup in Step 1315. Since the step-up process has been just started, the application voltage Vm is less than the upper limit voltage Vup. Accordingly, the CPU determines “YES” in Step 1315, and increases the application voltage Vm by a predetermined value based on the waveform illustrated in FIG. 5A in Step 1320. The CPU performs Step 1395.

(D) Immediately after the step-up process has been completed, it is assumed that it is after the step-up process has been just completed (that is, this routine is performed after the application voltage Vm is changed to the upper limit voltage Vup) and before the step-down process is started. In this case, the CPU determines “NO” in Step 1315, and decreases the application voltage Vm by a predetermined value based on the waveform illustrated in FIG. 5A in Step 1325 (that is, the step-down process is started). Subsequently, in Step 1330, the CPU determines whether the application voltage Vm is less than the current acquisition start voltage Vsem. Since the step-down process has been just started, the application voltage Vm is higher than the current acquisition start voltage Vsem. Accordingly, the CPU determines “NO” in Step 1330 and then performs Step 1395.

When this routine is performed next, the step-up process is not being performed (the step-down process is being performed), and thus the CPU determines “NO” in Step 1310 and performs Step 1325.

(E) When the application voltage Vm becomes less than the current acquisition start voltage Vsem by the step-down process, it is assumed that the application voltage Vm becomes less than the current acquisition start voltage Vsem and the application voltage Vm is higher than the lower limit voltage Vbt during the step-down process. In this case, the CPU determines “YES” in Step 1330, and determines whether the output current Im is less than the current minimum value Imin in Step 1335. When the output current Im is less than the current minimum value Imin, the CPU determines “YES” in Step 1335 and sets the current minimum value Imin as the output current Im in Step 1340. Subsequently, the CPU performs Step 1345.

On the other hand, when the output current Im is equal to or greater than the current minimum value Imin, the CPU determines “NO” in Step 1335 and then directly performs Step 1345.

In Step 1345, the CPU determines whether the application voltage Vm is equal to or less than the lower limit voltage Vbt. Since the application voltage Vm is higher than the lower limit voltage on the above-mentioned assumption, the CPU determines “NO” in Step 1345 and then performs Step 1395.

(F) When the application voltage Vm becomes equal to or less than the lower limit voltage Vbt by the step-down process, it is assumed that the application voltage Vm becomes equal to or less than the lower limit voltage Vbt by the step-down process. In this case, the CPU determines “YES” in Step 1345, and calculates (acquires) a difference between the reference current Ib and the current minimum value Imin as the current difference Idf in Step 1350. Subsequently, in Step 1355, the CPU determines whether the current difference Idf is equal to or greater than the current threshold value Ith.

When the current difference Idf is equal to or greater than the current threshold value Ith, the CPU determines “YES” in Step 1355, and determines that the SOx concentration in the exhaust gas is equal to or greater than a predetermined value in Step 1360 and writes the determination result to the backup RAM 34. In this case, the CPU turns on a warning lamp (not illustrated) in the vehicle in which the internal combustion engine 10 is mounted.

Subsequently, in Step 1365, the CPU sets the application voltage Vm to the oxygen concentration detection voltage Vmo. Then, in Step 1370, the CPU sets the value of the A/F detection flag Xaf to “1” and sets the value of the SOx determination flag Xsx to “0.” Subsequently, the CPU performs Step 1395.

On the other hand, when the determination condition in Step 1355 is not satisfied (that is, when the current difference Idf is less than the current threshold value Ith), the CPU determines “NO” in Step 1355, and determines that the SOx concentration in the exhaust gas is lower than a predetermined value and writes the determination result to the backup RAM 34 in Step 1362. Subsequently, the CPU performs Step 1365.

As described above, according to the present detection device, it is possible to prevent the output current Im from increasing rapidly due to the reductive decomposition of water in the step-up process and the step-down process and to prevent the value of the current minimum value Imin from change due to the reductive decomposition of water. Accordingly, according to the present detection device, it is possible to accurately determine whether the SOx concentration in the exhaust gas is higher than a predetermined threshold value. In addition, according to the present detection device, since the upper limit voltage Vup is not higher than necessary, it is possible to prevent the time required for the step-up process and the step-down process from becoming longer than necessary.

While a gas detection device according to an embodiment of the disclosure has been described above, the disclosure is not limited to the embodiment and can be modified in various forms without departing from the gist of the disclosure. For example, the ECU 30 according to the embodiment determines whether the SOx concentration in the exhaust gas is higher than a predetermined value by comparing the current difference Idf with the current threshold value Ith. However, the ECU 30 may detect (acquire) the SOx concentration in the exhaust gas. For example, the ECU 30 may detect the SOx concentration by applying the current difference Idf to a “map indicating a relationship between the current difference Idf and the SOx concentration” stored in the ROM 32. In this case, the ECU 30 detects the SOx concentration such that the SOx concentration increases as the current difference Idf increases.

The ECU 30 according to the embodiment acquires the output current Im when the application voltage Vm before the step-up process is started is equal to the oxygen concentration detection voltage Vmo as the reference current Ib. However, the reference current Ib may be another value (current value). For example, the reference current Ib may be an average value of the output currents Im in the step-up process and the step-down process. The reference current Ib may be set to the output current Im at a time point at which the step-up process is started (that is, when the application voltage Vm is equal to the lower limit voltage Vbt). The reference current Ib may be set to the output current Im at a time point at which the step-up process is ended (that is, when the application voltage Vm is equal to the upper limit voltage Vup). Alternatively, the reference current Ib may be set to the output current Im when the application voltage Vm in the step-up process and the step-down process is equal to a specific voltage.

The ECU 30 according to the embodiment determines whether the SOx concentration in the exhaust gas is higher than a predetermined value based on the current difference Idf. However, the ECU 30 may determine whether the SOx concentration in the exhaust gas is higher than a predetermined value based on the current minimum value Imin. Alternatively, the ECU 30 may detect (acquire) the SOx concentration by applying the current minimum value Imin to a “map indicating a relationship between the current minimum value Imin and the SOx concentration” stored in the ROM 32. In this case, the ECU 30 detects the SOx concentration such that SOx concentration increases as the current minimum value Imin decreases.

The first voltage and the third voltage in the embodiment are set to the same as the lower limit voltage Vbt. However, the first voltage and the third voltage may have different values.

The ECU 30 according to the embodiment sets the upper limit voltage Vup to become larger as the air-fuel ratio A/F of the engine becomes larger. However, this process may be skipped. That is, the ECU 30 may not change the upper limit voltage Vup with the air-fuel ratio A/F of the engine. On the other hand, the ECU 30 according to the embodiment sets the upper limit voltage Vup to become smaller as the element temperature Tcl becomes higher. However, this process may be skipped. That is, the ECU 30 may not change the upper limit voltage Vup with the element temperature Tcl.

What is claimed is:

1. A gas detection device comprising:
an electrochemical cell including a solid electrolyte which has oxide ion conductivity, of which a first surface is exposed to an exhaust gas of an internal combustion engine introduced via diffusion resistor, and of which a second surface other than the first surface is exposed to atmospheric air, a first electrode which is disposed on the first surface of the solid electrolyte, and a second electrode which is disposed on the second surface of the solid electrolyte;
a voltage applying unit that applies an application voltage, which has a positive value when a potential of the second electrode is higher than a potential of the first electrode, between the second electrode and the first electrode;
a current detecting unit that detects an output current which is a current flowing between the first electrode and the second electrode;
a voltage control unit that performs a step-up process of gradually stepping up the application voltage from a predetermined first voltage lower than a specific voltage which is a voltage at which reductive decomposition of sulfur oxide contained in the exhaust gas occurs to a predetermined second voltage higher than the specific voltage and performs a step-down process of gradually stepping down the application voltage to a predetermined third voltage lower than the specific voltage after the step-up process has been completed; and
a measurement unit that determines whether a sulfur oxide concentration in the exhaust gas is higher than a predetermined value or detects the sulfur oxide concentration in the exhaust gas based on a specific parameter which has a correlation with a degree of change of the output current caused due to return of sulfur generated by reductive decomposition of the sulfur oxide to sulfur oxide on the first electrode during the step-down process,
wherein the voltage control unit sets the second voltage to a voltage lower than a voltage at which a rapid increase of the output current occurs due to reductive decomposition of water contained in the exhaust gas when the application voltage increases gradually.

2. The gas detection device according to claim 1, further comprising an air-fuel ratio acquiring unit that acquires an air-fuel ratio of an air-fuel mixture in a combustion chamber of the internal combustion engine,
wherein the voltage control unit sets the second voltage to become higher as the air-fuel ratio of the air-fuel mixture in the combustion chamber of the internal combustion engine acquired by the air-fuel ratio acquiring unit becomes higher.

3. The gas detection device according to claim 2, further comprising a temperature acquiring unit that acquires a temperature of the electrochemical cell,
wherein the voltage control unit sets the second voltage to become lower as the temperature of the electrochemical cell acquired by the temperature acquiring unit becomes higher.

4. The gas detection device according to claim 1, further comprising a temperature acquiring unit that acquires a temperature of the electrochemical cell,
wherein the voltage control unit sets the second voltage to become lower as the temperature of the electrochemical cell acquired by the temperature acquiring unit becomes higher.
